(19) United States
(12) Patent Application Publication
(10) Pub. No.: US 2024/0238357 A1
(43) Pub. Date: Jul. 18, 2024

(54) CANNABIS AND MUSHROOM COMPOUNDS INFUSED CONSUMABLES

(71) Applicant: Michael William Randel, Lakebay, WA (US)

(72) Inventor: Michael William Randel, Lakebay, WA (US)

(21) Appl. No.: 18/097,370

(22) Filed: Jan. 16, 2023

(51) Int. Cl. A61K 36/07; A23D 9/007; A23D 9/04; A23F 5/14; A23L 7/10; A23L 19/00; A23L 25/00; A23L 33/105; A61K 36/185

(52) U.S. Cl. CPC A61K 36/07; A23D 9/007; A23D 9/04; A23F 5/14; A23L 7/10; A23L 19/03; A23L 25/20; A23L 33/105; A61K 36/185

(57) ABSTRACT

The present disclosure is directed to methods of producing consumables infused with whole spectrum cannabis compounds and mushroom compounds, the consumables including coffee beans, cacao beans, raw popcorn kernels, raw and roasted nuts, raw and roasted seed, and dehydrated fruit. The consumables are infused in an overall multi-step process, in which a food grade oil is first infused with whole spectrum cannabis compounds and whole spectrum mushroom compounds; the infused food grade oil turn is used to infuse a consumable with whole spectrum cannabis compounds and whole spectrum mushroom compounds.

### FIELD OF THE INVENTION

The invention relates to whole spectrum cannabis compounds and whole spectrum mushroom compounds that are infused into a food grade oil, which in turn is used to infuse a variety of consumables with whole spectrum cannabis compounds and whole spectrum mushroom compounds.

### BACKGROUND

Cannabis has been used to alleviate stress and other illnesses caused by posttraumatic stress disorder, seizures, epilepsy, multiple sclerosis, and the like. Cannabis, commonly known as marijuana or hemp, is a genus of flowering plants that includes at least three species, Cannabis sativa, Cannabis indica, and Cannabis ruderalis.

Cannabis plants produce a variety of potentially useful or beneficial compounds, such as cannabinoids, which produce mental and physical effects when consumed. Cannabinoids are a chemical group or family of 21-carbon-containing terpenophenolic compounds produced by Cannabis species. Current estimates of the number of cannabinoids found in Cannabis species is well in excess of 100 different cannabinoids. Two of the most prominent cannabinoids are Cannabidiol (CBD) and Tetrahydrocannabinol (THC). In addition to CBD and THC, other cannabinoids such as cannabichromene (CBC), cannabigerol (CBG), cannabinol (CBN), and others are present in varying amounts in cannabis plant material.

Commonly consumed sources of cannabis compounds include extracts, oils, isolates, and the like from Cannabis species including marijuana, hemp, and industrial hemp, which contains a THC content of less than 0.3% of overall mass. While providing useful or beneficial effects to the user, such extracts, oils, and isolates are typically found to have undesirable tastes, flavors, odors, and/or other unfavorable attributes. Accordingly, addition of hemp compound containing extracts or isolates to food or beverage consumables imparts an undesirable taste, since the extracts or isolates typically have an undesirable taste.

Mushrooms are commonly consumed for their taste and flavor as well as for compounds they contain. A wide variety of mushrooms are consumed directly or as flavoring for other foods. In addition, there are mushrooms consumed for health effects and psychoactive effects. For example, lion's mane mushrooms are valued for their health benefits.

A class of mushrooms collectively known as psilocybin mushrooms and commonly referred to as magic mushrooms are widely consumed for their psychoactive and psychedelic effects. These mushrooms contain psilocybin, a naturally occurring psychedelic compound that is converted in the liver into psilocin, which produces a psychoactive effect. The composition of psilocybin mushrooms varies from one species to another, with varying amounts of psilocybin as well as other compounds including psilocin, norpsilocin, baeocystin, norbaeocystin, and aeruginascin. These other compounds may modify the effects of psilocybin mushrooms in a manner that makes the combination of compounds preferable to consumption of just psilocybin.

There are many methods of consuming psilocybin mushrooms to obtain a desired psychoactive effect (s). Psilocybin mushrooms are commonly eaten, either as fresh or dried mushrooms, which has the significant drawbacks of a poor, bitter taste, requirement of significant chewing of the mushrooms, and often, queasiness from consumption. Another common form of consumption is to make and drink a mushroom tea, which again can have an unpleasant taste. Yet another common form of consumption is to grind mushrooms and add mushroom powder to a variety of foods, which can be messy, coupled with an unpleasant taste. Other forms of consumption include mushroom chocolate and a technique called lemon tek, where psilocybin mushrooms are soaked in lemon juice or some other kind of acidic liquid.

To date, there are no known consumables that contain both cannabis compounds and mushroom compounds. Accordingly, there exists a need in the art for consumables that contain a combination of whole spectrum compounds from cannabis and whole spectrum compounds from mushrooms. The presently disclosed methods of preparing such consumables containing cannabis and mushroom compounds and the resultant consumables address this need.

### SUMMARY

The present disclosure provides for methods of producing orally ingestible consumables infused with whole spectrum cannabis compounds and whole spectrum mushroom compounds.

The overall method disclosed entails a multi-step process in which a food grade oil, such as coconut oil or other food grade oil, is infused with whole spectrum compounds from a species of Cannabis and whole spectrum compounds from mushrooms. Following infusion of the food grade oil with whole spectrum cannabis compounds and whole spectrum mushroom compounds, the infused food grade oil is used to infuse a given consumable with whole spectrum cannabis compounds and whole spectrum mushroom compounds. A cannabis and mushroom compounds infused consumable can be consumed directly or used to generate a cannabis and mushroom compounds infused beverage, such as coffee from infused coffee beans.

In a preferred embodiment, a food grade oil infused with whole spectrum cannabis compounds and mushroom compounds is produced by separately infusing a first food grade oil with cannabis compounds and a second food grade oil with mushroom compounds. The infused first food grade oil is then combined with the infused second food grade oil to yield a food grade oil infused with whole spectrum cannabis compounds and whole spectrum mushroom compounds. This infused food grade oil is in turn used to infuse a given consumable with whole spectrum cannabis compounds and whole spectrum mushroom compounds.

The presently disclosed methods of infusing a food grade oil with whole spectrum cannabis compounds and/or whole spectrum mushroom compounds involve a series of steps in which starting cannabis material and/or starting mushroom material is mixed with a food grade oil followed by steps of heating and freezing the mixture of starting material and food grade oil. The mixture of starting material and food grade oil is subsequently strained to separate the starting material from the food grade oil to yield a food grade oil infused with whole spectrum cannabis compounds and/or whole spectrum mushroom compounds.

In an alternative embodiment, a food grade oil is first infused with whole spectrum cannabis compounds, and that infused food grade oil is then infused with whole spectrum mushroom compounds to yield a food grade oil infused with whole spectrum cannabis compounds and whole spectrum mushroom compounds.

In another alternative embodiment, a food grade oil is first infused with whole spectrum mushroom compounds, and that infused food grade oil is then infused with whole spectrum cannabis compounds to yield a food grade oil infused with whole spectrum cannabis compounds and whole spectrum mushroom compounds.

In yet another embodiment, a food grade oil is simultaneously infused with whole spectrum cannabis compound and whole spectrum mushroom compounds by infusing a food grade oil with a combination of starting cannabis material and mushroom material to yield a food grade oil infused with whole spectrum cannabis compounds and whole spectrum mushroom compounds.

Following the production of a food grade oil infused with whole spectrum cannabis compounds and whole spectrum mushroom compounds, the infused food grade oil is used to infuse consumables with whole spectrum cannabis compounds and whole spectrum mushroom compounds. As detailed herein, the method involves a series of steps of heating and freezing consumables in the presence of a food grade oil infused with whole spectrum cannabis compounds and whole spectrum mushroom compounds. The mixture of consumables and infused food grade oil is subsequently strained to separate the consumables from the infused food grade oil to yield consumables infused with whole spectrum cannabis compounds and whole spectrum mushroom compounds.

The presently disclosed consumables infused with whole spectrum cannabis compounds and whole spectrum mushroom compounds are more fully described in the detailed description below.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 shows example steps of a method according to exemplary embodiments hereof.

FIG. 2 shows example steps of a method according to exemplary embodiments hereof.

FIG. 3 shows example steps of a method according to exemplary embodiments hereof.

FIG. 4 shows example steps of a method according to exemplary embodiments hereof.

FIG. 5 shows example steps of a method according to exemplary embodiments hereof.

FIG. 6 shows example steps of a method according to exemplary embodiments hereof.

FIG. 7 shows example steps of a method according to exemplary embodiments hereof.

### DETAILED DESCRIPTION OF EXEMPLARY EMBODIMENTS

As is known in the art, plants in the cannabis family (e.g., hemp, marijuana, etc.) produce a number of beneficial compounds, including cannabinoids. To date, more than 100 cannabinoids have been identified, accounting for up to 40% of the plant's extract. Well known cannabinoids include Cannabidiol (CBD), Tetrahydrocannabinol (THC) among others, each of which may include a variety of health benefits.

Mushrooms are commonly consumed for their flavors, health benefits, and psychoactive effects. The flavors, health benefits, and psychoactive effects of mushrooms can be attributed to one or more compounds found in mushrooms.

The presently disclosed combination of cannabis compounds and mushroom compounds infused consumables and methods of producing such consumables provide for a wide range of food and beverage consumables that impart desired qualities and effects as a result of combined, infused cannabis and mushroom compounds in the consumables.

The cannabis and mushroom compounds infusion methods described herein can be used to infuse a wide variety of food consumables, which in some instances can in turn be used to make an infused beverage. Virtually any food consumable that can be effectively manipulated in the method(s) described herein can be a candidate for infusion with cannabis and mushroom compounds. Examples of such foods include, but are not limited to, coffee beans, cacao beans, raw nuts and seeds, roasted nuts and seeds, popcorn kernels, dehydrated fruit, raw fruit, trail mix, granola bars, garlic, beans, bacon, ice cream, jams and jellies, baked goods, caramel sauce, and freeze-dried consumables.

As used herein, the terms “infused cannabis compounds and mushroom compounds” or “cannabis and mushroom compounds infused” refers to consumables to which whole spectrum compounds from cannabis and mushrooms have been infused by a method(s) disclosed herein. The infusion method generally involves preparing a food grade oil (such as coconut oil or a similar food grade oil) that is infused with whole spectrum cannabis compounds and whole spectrum mushroom compounds. The food grade oil infused with whole spectrum cannabis and mushroom compounds is then used to infuse consumables as detailed herein. Throughout this specification, cannabis and mushroom compounds infused food and/or drink may be referred to as “cannabis and mushroom compounds infused” or simply “infused,” such as “cannabis and mushroom compounds infused cacao beans” or “infused cacao beans.”

A food grade oil infused with whole spectrum cannabis and mushroom compounds may be prepared using any of a number of approaches. In a preferred embodiment, separate cannabis compounds and mushroom compounds infused food grade oils are initially prepared and then combined to yield a food grade oil infused with whole spectrum cannabis compounds and whole spectrum mushroom compounds. In an alternative embodiment, a food grade oil is initially infused with whole spectrum cannabis compounds, and then that cannabis compounds infused food grade oil is in turn infused with whole spectrum mushroom compounds to yield a food grade oil infused with whole spectrum cannabis compounds and whole spectrum mushroom compounds. In another embodiment, a food grade oil is initially infused with whole spectrum mushroom compounds, and then that mushroom compounds infused food grade oil is in turn infused with whole spectrum cannabis compounds to yield a food grade oil infused with whole spectrum cannabis compounds and whole spectrum mushroom compounds. In yet another embodiment, a food grade oil is infused with mixture of whole cannabis plant materials and mushroom materials (whole or parts of mushrooms) to yield a food grade oil infused with whole spectrum cannabis compounds and whole spectrum mushroom compounds.

The infusion method generally involves preparing a mixture made with stating material (such as cannabis plant materials and/or mushroom materials) and a food grade oil (such as coconut oil or a similar food grade oil) under specific heating and cooling conditions to produce a whole spectrum compounds (cannabis, mushroom, or both) infused oil. After a cannabis and mushroom whole spectrum compounds infused oil is produced, the infused oil is then used to infuse consumables as detailed herein.

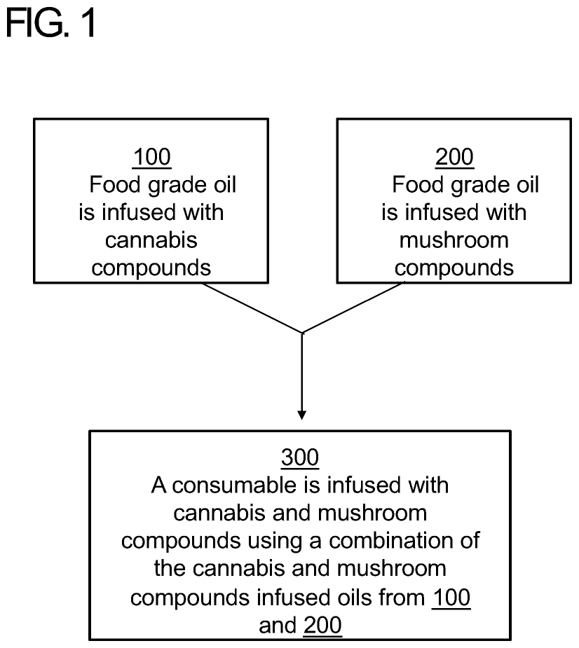

In some embodiments as shown in FIG. 1, cannabis compounds and mushroom compounds infused foods and/or drinks prepared by the methodology disclosed herein are generated in an overall three-step process. In this process, independent cannabis whole spectrum and mushroom whole spectrum infused food grade oils are produced separately and then combined to yield an oil infused with whole spectrum cannabis compounds and whole spectrum mushroom compounds. In the first step (at 100), whole cannabis plant material, preferably small buds, is used to infuse a food grade oil, such as coconut oil, with cannabis compounds. Alternatively, whole cannabis plant material that has been ground can be used to infuse an oil. Note that because whole cannabis plant material is used at 100, the resulting infusion of the oil from the cannabis provides a full spectrum of compounds from the cannabis infused into the oil. The infused oil can also be regarded as a cannabis extract. In a second step (at 200), mushroom material (whole mushrooms or parts of mushrooms) is used to infuse a food grade oil, such as coconut oil, with mushroom compounds. Note that because whole mushroom material (whether whole or parts of mushrooms) is used at 200, the resulting infusion of the oil from the mushroom material provides a full spectrum of mushroom compounds from the mushroom material infused into the oil. The infused oil can be regarded as a mushroom extract. In a third step (at 300), cannabis compounds infused food grade oil is combined with mushroom compounds infused food grade oil, and the combination infused food grade oil is used to infuse an end food product/consumable with whole spectrum cannabis and mushroom compounds. After the end product is infused with whole spectrum cannabis and mushroom compounds, it may be consumed in any typical fashion, such as direct consumption, used to make a beverage as in the case of coffee or cacao beans, and so forth.

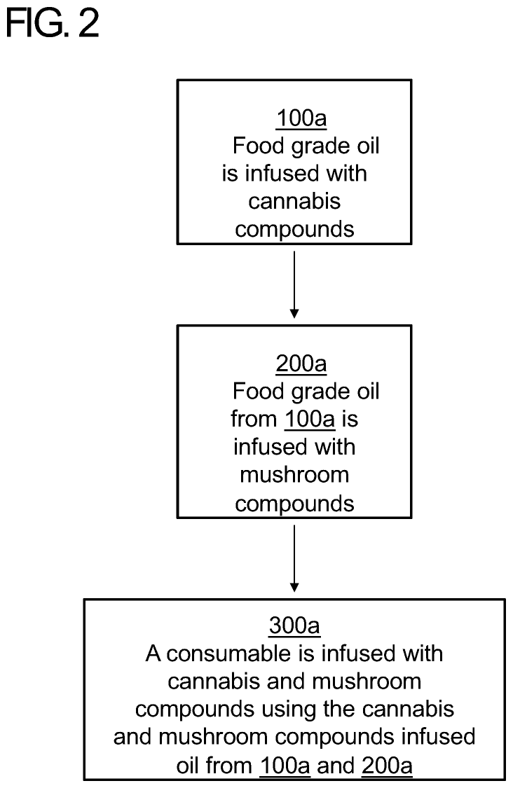

In an alternative embodiment, cannabis compounds and mushroom compounds infused foods and/or drinks prepared by the methodology disclosed herein are generated in another overall three-step process as shown in FIG. 2. In the first step (at 100a), a food grade oil is infused with whole spectrum cannabis compounds, as described above. This cannabis compounds infused food grade oil is next infused with whole spectrum mushroom compounds (at 200a) to yield a food grade oil infused with whole spectrum cannabis compounds and whole spectrum mushroom compounds. In a third step (at 300a), the infused food grade oil is used to infuse an end food product/consumable with whole spectrum cannabis compounds and whole spectrum mushroom compounds.

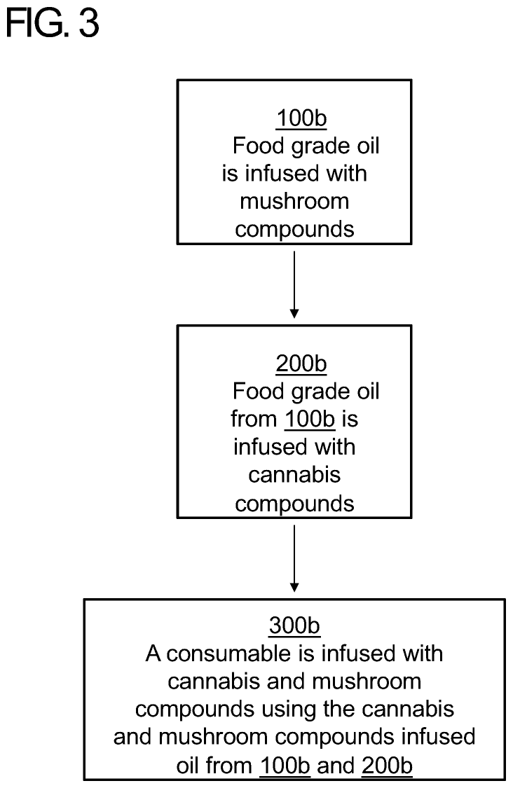

In a related alternative embodiment, cannabis compounds and mushroom compounds infused foods and/or drinks prepared by the methodology disclosed herein are generated in another overall three-step process as shown in FIG. 3. In the first step (at 100b), a food grade oil is infused with whole spectrum mushroom compounds, as described above. This mushroom compounds infused food grade oil is next infused with whole spectrum cannabis compounds (at 200b) to yield a food grade oil infused with whole spectrum cannabis compounds and whole spectrum mushroom compounds. In a third step (at 300b), the infused food grade oil is used to infuse an end food product/consumable with whole spectrum cannabis compounds and whole spectrum mushroom compounds.

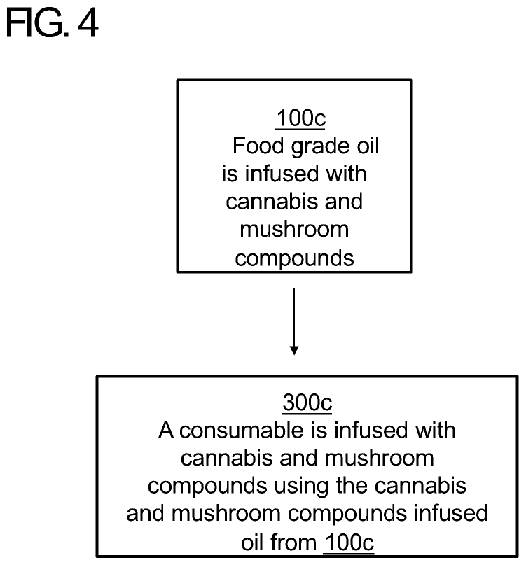

In yet another alternative embodiment, cannabis compounds and mushroom compounds infused foods and/or drinks prepared by the methodology disclosed herein are generated in an overall two-step process as shown in FIG. 4. In the first step (at 100c), whole cannabis plant material, preferably small buds, is combined with mushroom material (whole mushrooms or parts of mushrooms) and the combined materials are used to infuse a food grade oil, preferably coconut oil, with cannabis and mushroom compounds. Alternatively, whole cannabis plant material that has been ground can be used in place of buds. Note that because whole cannabis plant and mushroom materials are used, the resulting infusion of the food grade oil from the cannabis and mushroom materials provides a full spectrum of compounds from the cannabis and mushroom materials infused into the oil. The infused oil can be regarded as a cannabis and mushroom extract. In a second step (at 300c), cannabis and mushroom compounds infused food grade oil is used to infuse an end food product/consumable with whole spectrum cannabis compounds and whole spectrum mushroom compounds. After the end product is infused with whole spectrum cannabis compounds and mushroom compounds, it may be consumed in any typical fashion, such as direct consumption, used to make a beverage as in the case of coffee or cacao beans, and so forth.

Consumption of the cannabis and mushroom compounds infused food and drink consumables generated by the methods described herein provides the beneficial effects generally associated with cannabis and mushrooms. These cannabis effects include, but are not limited to, mental and physical effects, such as pain relief from CBDs and other cannabinoids, mental high from THC (in foods infused with marijuana cannabinoids), and other effects attributed to consumption of cannabinoids. Likewise, these mushroom effects include, but are not limited to, food flavoring, mental and physical effects, psychoactive effects (such as in foods infused with psilocybin containing mushrooms), and other effects attributed to consumption of mushrooms.

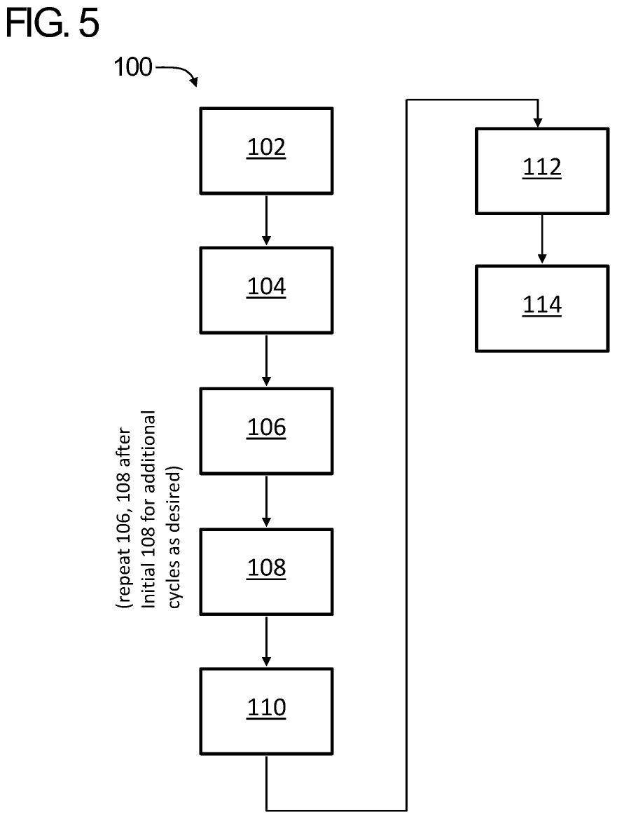

FIG. 5 refers to steps that may be taken to infuse a food grade oil with cannabis compounds to complete step 100 of FIG. 1. Suitable food grade oils for this process include, but are not limited to, coconut oil, medium chain triglycerides (MCT) oil, vegetable oil, sunflower oil, grape seed oil, olive oil, avocado oil, and combinations thereof. Likewise, butter, shortening, margarine, or lard may be used in the process.

In one embodiment as shown in FIG. 5, whole, raw cannabis plant material (e.g., flowers, buds, leaves, or other plant material; preferably small buds) is frozen (at 102). The cannabis plant material can be used whole or ground (which is preferably produced by grinding the frozen plant material). The cannabis plant may include Cannabis Sativa, Cannabis Indica, Cannabis Ruderalis, other types of cannabis and any combination thereof. The cannabis may be classified as marijuana, hemp, and/or other types of cannabis.

The cannabis plant material may also be a strain that is particularly rich in one or more specific cannabinoids, such as a strain that is particularly rich in cannabidiol (CBD) or cannabigerol (CBG). In addition, the cannabis plant material may be a combination of two or more different strains that are particularly rich for a specific cannabinoid. Currently available cannabis strains are typically rich in CBD, but strains that are rich for other cannabinoids have been and are being developed. For example, more recently developed strains include those that are rich in CBG. Generally, cannabigerol (CBG) and cannabigerolic acid (CBGA) only account for 1% of a hemp strain's total cannabinoids. In a CBG-rich hemp strain, cannabigerol makes up around 10-15% or more of the cannabis flower's total cannabinoids. In a preferred embodiment, the cannabis plant material used in the presently disclosed method(s) is a mixture of equal parts of two strains that are rich in CBD and CBG, respectively. Additional strains that are particularly rich in a specific cannabinoid and combinations thereof fall within the scope of the presently disclosed method(s).

Next (at 104), a suitable food grade oil such as coconut oil, MCT oil, a combination of coconut oil and MCT oil (or similar oil as described above) is heated to a temperature equal to or between 1700 and 240° F., and preferably to about 185° F. For the purposes of this specification, the term “about” used in relation to temperatures will mean within ±1%. Regarding a combination of coconut oil and MCT oil, a preferred embodiment is to use a ratio of 75% coconut oil to 25% MCT oil. Alternatively, ratios of coconut oil to MCT oil can range from essentially any ratio of the two oils, such as, but not limited to, 90% coconut oil:10% MCT to 10% coconut oil: 90% MCT oil.

Next (at 106), the frozen cannabis (preferably small buds) is added to the food grade oil and held at the temperature between 170° and 240° F. (e.g., preferably at about 185° F.) for 4-12 hours (preferably 5-7 hours), while occasionally (or continuously) stirring the mixture. In some embodiments, the ratio of plant material to coconut oil is 1 lb plant material to 2 gallons oil. For example, on a commercial scale, 100 lbs plant material/buds may be mixed with 200 gallons of oil. However, other ratios within 10%, 20%, 30%, 40%, 50%, 75%, 100% of this ratio also may be used. In general, the ratio will be chosen to provide high levels/concentration(s) of cannabis compounds within the oil.

After the heating step of 106, in the next step (at 108), the cannabis and food grade oil mixture is frozen to obtain a solid, frozen biomass of cannabis and food grade oil mixture.

At this juncture, the solid, frozen biomass of cannabis and food grade oil can be processed in one of several ways. First, the cannabis and food grade oil can be heated and strained as per steps 110, 112, and 114 to yield cannabis compounds infused food grade oil produced by a single round or phase of heating and freezing. Second, the cannabis and food grade oil alternatively can be treated with a second round or phase of heating and freezing, in which steps 106 and 108 are repeated, followed by subsequent steps 110, 112, and 114 to yield cannabis compounds infused food grade oil produced by two rounds of heating and freezing. Third, the cannabis and food grade oil alternatively can be treated with a third round or phase of heating and freezing, in which steps 106 and 108 are repeated twice, followed by subsequent steps 110, 112, and 114 to yield cannabis compounds infused food grade oil produced by three rounds of heating and freezing The single round/phase treatment will produce cannabis compounds infused food grade oil having a lesser relative level of infused cannabis compounds compared to a double round/phase treatment. While the double round/phase treatment has been found to provide preferable levels of cannabis compounds infused into the food grade oil, in some instances, a third round/phase treatment may be more preferable. The single round/phase treatment can produce cannabis compounds-infused food grade oil that is adequate for many applications. If higher infused levels of cannabis compounds in food grade oil are desired for a particular application, the double round/phase treatment or even triple round/phase treatment of cannabis compounds infused food grade oil can be applied.

Following step 108, at step 110, the solid, frozen cannabis and food grade oil biomass is reheated to 150° to 200° F., and preferably to about 1850 F, and held at the temperature for a sufficient period of time to liquefy the mixture, typically for about 2-4 hours.

Next (at 112), the cannabis and food grade oil mixture is strained using a press bag (or other suitable straining techniques) to separate the cannabis compounds infused food grade oil from the cannabis biomass. Note that the cannabis compounds infused food grade oil can also be referred to as a cannabis extract.

In a further step (114), the strained, cannabis compounds infused food grade oil is heated to between 250° F. to 350° F. for 3-6 hours. This step will decarboxylate the carboxylated cannabinoids in the oil, such as CBD-A or THC-A. The temperature and time will vary based in part on the cannabis strain used and desired decarboxylation. For example, using a CBD-rich strain that provides high levels of CBD-A, the step will preferably include a temperature of 2500 F for 3-6 hours. In another example, using a CBG-rich strain that provides high levels of CBG-A, the step will preferably include a temperature of 300° F. for 3-6 hours.

This method results in a whole spectrum cannabis compounds infused food grade oil that may be used in combination with a whole spectrum mushroom compounds infused food grade oil for infusing consumables with whole spectrum cannabis compounds and whole spectrum mushroom compounds as described below.

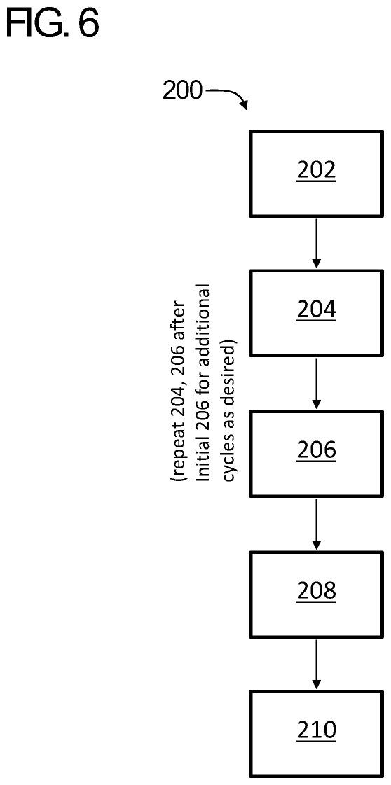

FIG. 6 refers to steps that may be taken to infuse a food grade oil with mushroom compounds to complete step 200 of FIG. 1. Suitable food grade oils for this process include, but are not limited to, coconut oil, medium chain triglycerides (MCT) oil, vegetable oil, sunflower oil, grape seed oil, olive oil, avocado oil, and combinations thereof. Likewise, shortening, margarine, butter, or lard may be used in the process.

In one embodiment as shown in FIG. 6, whole or parts of mushrooms can be used in the process, depending on the desired infusion effects. Parts of mushrooms may have a higher concentration of a desired, specific compound or compounds, such that the most effective infusion occurs with a part of a mushroom (rather than whole mushrooms). Accordingly, it may be preferable to use just mushroom caps, stalks/stems, mycelium, combinations thereof, and so forth in the presently described method(s). For example, a particular species of psilocybin mushroom might contain higher concentrations of psilocybin in the cap as opposed to the stem, such that it's advantageous to conduct the infusion with just caps, rather than the whole mushroom.

Virtually any type, variety, or species of mushroom can be used in the presently described method(s). For example, infusion of mushroom flavors can be obtained from any particular mushroom variety typically consumed for its flavor or in cooking, such as, but not limited to Chanterelle, Truffle—Black & White, Morel, Trumpet—King & Black, Oyster, Shiitake, Maitake—Hen of the Woods, Cremini, Lobster, Porcini, Hedgehog, Portobello, Button/White, Enoki, Beech, and combinations thereof. Infusion of compounds that may help reduce the effects of stress on one's body can be obtained from adaptogenic mushrooms, such as Cordyceps, Lion's Mane, and Reishi mushrooms. Likewise, exemplary species of mushrooms recognized for exemplary beneficial effects and function can include, but are not limited to, Argarikon (immune response), Cordyceps (energy, stamina, and libido), Chaga (antioxidants, immunity balancer, anti-inflammatory, and energy), Lion's Mane (cognitive function, concentration, focus, and sleep), Maitake (immunity booster and anti-inflammatory), Reishi (immunity booster, stress and anxiety relief, and sleep), Turkey Tail (immune function and gut), Shiitake (immunity booster, hair, skin and nails), and combinations thereof. In addition, there are well over 100 species of psilocybin mushrooms that contain psilocybin, which is a known psychoactive and psychedelic compound that may have beneficial effects with respect to treating addiction, depression, death anxiety, anorexia, demoralization syndrome, and obsessive-compulsive disorder. Accordingly, infusion of food and beverage with mushroom compounds can provide a wide range of desired properties, benefits, and function, such as flavoring, functional, and psychoactive properties for addressing anxiety, depression, addiction, additional mental illnesses, and so forth.

As shown in FIG. 6 (at 202), a suitable food grade oil such as coconut oil, MCT oil, another similar oil as described above, a combination of coconut oil and MCT oil, or a combination of two or more similar oils as described above, is heated to a temperature equal to or between about 170° and 240° F. In a preferred embodiment, the temperature is equal to or between about 1800 and 220° F. For the purposes of this specification, the term “about” used in relation to temperatures will mean within ±1%. Regarding a combination of coconut oil and MCT oil, a preferred embodiment is to use a ratio of 75% coconut oil to 25% MCT oil. Alternatively, ratios of coconut oil to MCT oil can range from any ratio of coconut oil to MCT oil, such as, but not limited to, 90% coconut oil:10% MCT oil to 10% coconut oil: 90% MCT oil.

Next (at 204), the mushroom material (whole or parts of mushrooms) is added to the food grade oil and held at the temperature between 170° and 240° F. for 4-12 hours, while occasionally (or continuously) stirring the mixture. In some embodiments, the ratio of mushroom material to good grade oil is 1 lb. mushroom material to 2 gallons oil. However, other ratios within 10%, 20%, 30%, 40%, 50%, 75%, 100% of this ratio also may be used. In general, the ratio will be chosen to provide high levels/concentration(s) of mushroom compounds within the oil.

After the heating step of 204, in the next step (at 206), the mushroom material and food grade oil mixture is frozen to obtain a solid, frozen biomass of mushroom material and food grade oil mixture.

At this juncture, the solid, frozen biomass of mushroom material and food grade oil can be processed in one of several ways. First, the frozen biomass can be heated and strained as per steps 208 and 210 (below) to yield mushroom compounds infused food grade oil produced by a single cycle or phase of heating and freezing. Second, the frozen biomass alternatively can be treated with a second cycle or phase of heating and freezing, in which steps 204 and 206 are repeated, followed by subsequent steps 208 and 210 to yield mushroom compounds infused food grade oil produced by two cycles of heating and freezing. Third, the second cycle treatment can be followed by a third cycle or phase of heating and freezing, in which steps 204 and 206 are repeated twice, followed by subsequent steps 208 and 210 to yield mushroom compounds infused food grade oil produced by three cycles of heating and freezing. Fourth, following a third cycle, the frozen biomass can be treated by a fourth cycle or phase of heating and freezing, in which steps 204 and 206 are repeated three times, followed by heating the frozen biomass and straining it as per steps 208 and 210 to yield mushroom compounds infused food grade oil produced by four cycles or phases of heating and freezing.

The single cycle/phase treatment typically will produce mushroom compounds infused food grade oil having a lesser relative level of infused mushroom compounds compared to a double cycle/phase treatment that will typically yield a greater level of infused mushroom compounds. For some particular applications, a third and/or fourth cycle treatment may yield an even greater level of infused mushroom compounds than a single or double cycle treatment. In a preferred embodiment, a three-cycle treatment is used.

Following step 206, at step 208, the solid, frozen mushroom and food grade oil biomass is reheated to 150° to 200° F. and held at the temperature for a sufficient period of time to liquefy the mixture, typically for about 2-4 hours.

Next (at 210), the mushroom and food grade oil mixture is strained using a press bag (or other suitable straining techniques) to separate the mushroom compounds infused food grade oil from the mushroom biomass.

This method results in a whole spectrum mushroom compounds infused food grade oil that may be used in combination with a whole spectrum cannabis compounds infused food grade oil for infusing consumables, as described below. The infused food grade oil may be referred to as a mushroom extract.

As detailed below, cannabis compounds and mushroom compounds infused food grade oil is used to infuse consumables with whole spectrum cannabis compounds and whole spectrum mushroom compounds. In a preferred embodiment, independently produced cannabis compounds infused food grade oil is combined with independently produced mushroom compounds infused food grade oil, and the combination is used to infuse consumables (as outlined in FIG. 1). In this scenario, the two separate infused oils can be combined in any of a range of ratios to produce desired infused levels of cannabis and mushroom compounds in consumables. Such ratios include, but are not limited to, 90% cannabis compounds infused food grade oil:10% mushroom compounds infused food grade oil to 10% cannabis compounds infused food grade oil:90% mushroom compounds infused food grade oil. In a preferred embodiment, an equal ratio of 50%:50% of each infused food grade oil is used.

In an alternative embodiment, the cannabis and mushroom compounds infused food grade oil is produced by first infusing an oil with cannabis compounds, and then infusing that cannabis compounds infused oil with mushroom compounds to generate a cannabis and mushroom compounds infused food grade oil (as outlined in FIG. 2). In this scenario, cannabis compounds infused food grade oil is produced as described above and outlined in FIG. 5. The cannabis compounds infused food grade oil is then infused with mushroom compounds following the methods described above for infusing a food grade oil with mushroom compounds and outlined in FIG. 6. This overall two-step process of first infusing food grade oil with cannabis compounds followed by a second infusion of the same oil with mushroom compounds will produce a whole spectrum cannabis compounds and whole spectrum mushroom compounds infused food grade oil.

In another alternative embodiment, the cannabis and mushroom compounds infused food grade oil is produced by first infusing an oil with mushroom compounds, and then infusing that mushroom compounds infused oil with cannabis compounds to generate a cannabis and mushroom compounds infused food grade oil (as outlined in FIG. 3). In this scenario, mushroom compounds infused food grade oil is produced as described above and outlined in FIG. 6. The mushroom compounds infused food grade oil is then infused with cannabis compounds following the methods described above for infusing a food grade oil with cannabis compounds and outlined in FIG. 5. This overall two-step process of first infusing food grade oil with mushroom compounds followed by a second infusion of the same oil with cannabis compounds will produce a whole spectrum cannabis compounds and whole spectrum mushroom compounds infused food grade oil.

In yet another alternative embodiment, the cannabis and mushroom compounds infused food grade oil is produced in an overall one-step process by simultaneously infusing an oil with cannabis and mushroom compounds, as outlined in FIG. 4. In this scenario, a cannabis and mushroom compounds infused food grade oil is produced by combining cannabis and mushroom materials and then infusing a food grade oil. The process for infusing food grade with combined cannabis and mushroom materials can be performed as described for producing cannabis infused food grade oil and outlined in FIG. 5 or can be performed as described for producing mushroom infused food grade oil and outlined in FIG. 6. Preferably, a food grade oil produced in the overall one-step process using combined cannabis and mushroom starting material is produced by the process described above for producing cannabis infused food grade oil and outlined in FIG. 5.

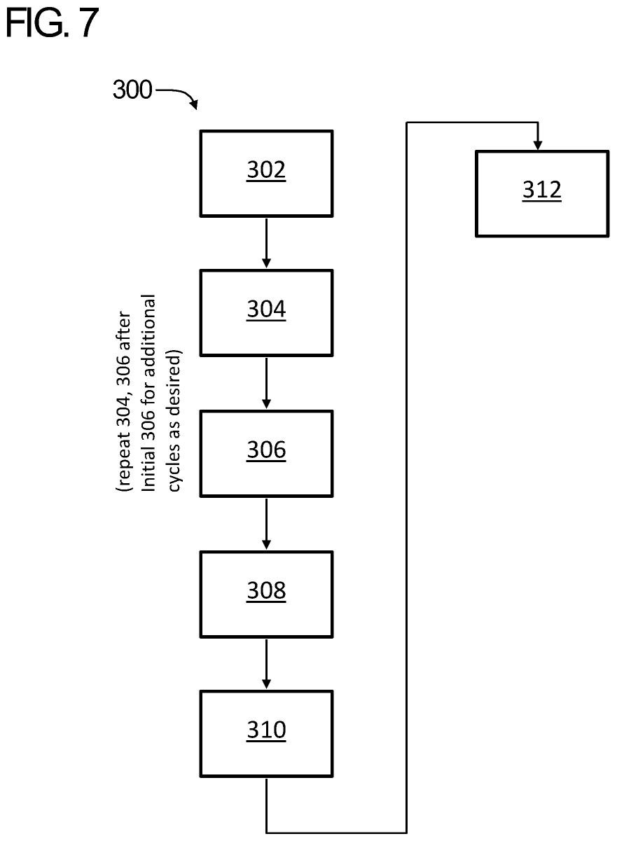

After a cannabis and mushroom whole compounds infused food grade oil has been produced, in an embodiment outlined in FIG. 7, the whole spectrum cannabis compounds and whole spectrum mushroom compounds infused food grade oil is used to infuse consumables, such as, but not limited to, coffee beans, cacao beans, dehydrated fruit, and raw popcorn kernels with whole spectrum cannabis and mushroom compounds.

First (at 302), at least a portion of the cannabis and mushroom compounds infused oil (as produced above) is heated to a first temperature between about 170° F. and 240° F.

Next (at 304), a selection of consumables is added and stirred into the infused food grade oil (preferably with continuous stirring) and held at the first temperature of about 170° F. to 240° F. for about 4-12 hours, with a preferred inner time range of 5-6 hours. For the purposes of this specification, the term “about” used in relation to periods of time will mean±3%.

Next (at 306), the mixture of the consumable and infused oil is frozen into a solid, frozen biomass.

At this juncture, the solid, frozen biomass of infused food grade oil and consumable can be processed in one of several ways. First, the infused food grade oil and consumable can be heated, strained, and frozen or packaged as per steps 308, 310, and 312 (below) to yield a cannabis and mushroom compounds infused consumable produced by a single round or phase of heating and freezing. Second, the infused food grade oil and consumable alternatively can be treated with a second cycle or phase of heating and freezing, in which steps 304 and 306 are repeated, followed by subsequent steps 308, 310, and 312 to yield a cannabis and mushroom compounds infused consumable produced by two cycles of heating and freezing. Third, the second cycle treatment can be followed by a third cycle or phase of heating and freezing, in which steps 304 and 306 are repeated twice. Following a third cycle, the frozen biomass can be heated and strained as per steps 308 and 310 to yield a cannabis and mushroom compounds infused consumable produced by three cycles or phases of heating and freezing.

The single cycle/phase treatment typically will produce a cannabis and mushroom compounds infused consumable having a lesser relative level of infused cannabis and mushroom compounds compared to a double cycle/phase treatment that will typically yield a greater level of infused mushroom compounds. For some particular applications and desired infusion levels, a third cycle treatment may yield an even greater level of infused cannabis and mushroom compounds than a single or double cycle treatment.

Following step 306, at step 308, the mixture including the now infused consumable and infused food grade oil is heated to a temperature equal to or between 150° F. and 200° F. and held at the temperature for a sufficient period of time to liquefy the food grade oil, typically about 2-4 hours, and preferably for about 2 hours.

After this heating step at 308, the mixture is strained at step 310 (using any suitable straining techniques) and the whole spectrum cannabis compounds and whole spectrum mushroom compounds infused consumables are removed from the food grade oil.

Then (at 312), the whole spectrum cannabis compounds and whole spectrum mushroom compounds infused consumables are typically frozen and stored frozen for subsequent use/packaging/consumption. Alternatively, the cannabis and mushroom compounds infused consumables can be directly used, packaged, or consumed without freezing.

This process 302-312 (FIG. 7) results in a food consumable infused with whole spectrum cannabis compounds and whole spectrum mushroom compounds, which can be referred to as a cannabis and mushroom compounds infused consumable.

In some embodiments, the freezing step at 306 provides organic pressure to the coffee beans, cacao beans, dehydrated fruit, raw popcorn kernels, or other consumables that deepens the physical depth of the cannabis and mushroom compounds infusion into the consumables. For example, in some embodiments, the freezing step at 306 causes the cannabis compounds and mushroom compounds to be pressed 10%, 20%, 30%, 40%, 50%, 60%, 70%, 80%, 90% and/or 100% to the centers of the coffee beans, cacao beans, dehydrated fruit, raw popcorn kernels, or other consumables.

It is understood that the acts described above are meant as a general overview and demonstration of an exemplary method, and that the method may include different and/or additional acts as described herein or otherwise.

While the present invention has been described as having particular configurations disclosed herein, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

It is understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (e.g., a step is performed by or with the assistance of a human).

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, term “at least one” should be understood as meaning “one or more”, and therefore includes both embodiments that include one or multiple components. Furthermore, dependent claims that refer to independent claims that describe features with “at least one” have the same meaning, both when the feature is referred to as “the” and “the at least one”.

As used in this description, the term “portion” means some or all. So, for example, “A portion of X” may include some of “X” or all of “X”. In the context of a conversation, the term “portion” means some or all of the conversation.

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

It should be appreciated that the words “first,” “second,” and so on, in the description and claims, are used to distinguish or identify, and not to show a serial or numerical limitation. Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish and/or identify, and are not intended to be otherwise limiting or to impose or imply any serial or numerical limitations or orderings. Similarly, words such as “particular,” “specific,” “certain,” and “given,” in the description and claims, if used, are to distinguish or identify, and are not intended to be otherwise limiting.

As used herein, including in the claims, the terms “multiple” and “plurality” mean “two or more,” and include the case of “two.” Thus, e.g., the phrase “multiple ABCs,” means “two or more ABCs,” and includes “two ABCs.” Similarly, e.g., the phrase “multiple PQRs,” means “two or more PQRs,” and includes “two PQRs.”

The present invention also covers the exact terms, features, values and ranges, etc. in case these terms, features, values and ranges etc. are used in conjunction with terms such as about, around, generally, substantially, essentially, at least etc. (i.e., “about 3” or “approximately 3” shall also cover exactly 3 or “substantially constant” shall also cover exactly constant).

As used herein, including in the claims, singular forms of terms are to be construed as also including the plural form and vice versa, unless the context indicates otherwise. Thus, it should be noted that as used herein, the singular forms “a,” “an,” and “the” include plural references unless the context clearly dictates otherwise.

Throughout the description and claims, the terms “comprise”, “including”, “having”, and “contain” and their variations should be understood as meaning “including but not limited to”, and are not intended to exclude other components unless specifically so stated.

It will be appreciated that variations to the embodiments of the invention can be made while still falling within the scope of the invention. Alternative features serving the same, equivalent or similar purpose can replace features disclosed in the specification, unless stated otherwise. Thus, unless stated otherwise, each feature disclosed represents one example of a generic series of equivalent or similar features.

The present invention also covers the exact terms, features, values and ranges, etc. in case these terms, features, values and ranges etc. are used in conjunction with terms such as about, around, generally, substantially, essentially, at least etc. (i.e., “about 3” shall also cover exactly 3 or “substantially constant” shall also cover exactly constant).

Use of exemplary language, such as “for instance”, “such as”, “for example” (“e.g.,”) and the like, is merely intended to better illustrate the invention and does not indicate a limitation on the scope of the invention unless specifically so claimed.

While the invention has been described in connection with what is presently considered to be the most practical and preferred embodiments and is further described in the examples below, it is to be understood that the invention is not to be limited to the disclosed embodiment, but on the contrary, is intended to cover various modifications and equivalent arrangements included within the spirit and scope of the appended claims.

### Example I: Coffee Beans

Cannabis and mushroom compounds infused roasted coffee beans may be made by infusing roasted coffee beans with a cannabis and mushroom compounds infused food grade oil obtained as described above and outlined in FIGS. 1-7.

In a preferred embodiment, cannabis and mushroom compounds infused roasted coffee beans may be made by infusing roasted coffee beans with a cannabis and mushroom compounds infused food grade oil as described in the process 302-212 (FIG. 7). The heating steps described in the process 302-312 (FIG. 7) for infusing roasted coffee beans are typically carried out at a temperature between 175° F. and 185° F. Once the cannabis and mushroom compounds infused roasted coffee beans are obtained, they can be stored frozen to help preserve freshness and slow/prevent deterioration of the cannabis and mushroom compounds that reduces the efficacy of the compounds. The infused roasted coffee beans can be used to make cannabis and mushroom compounds infused coffee beverage(s).

### Example II: Cacao Beans

The process by which cacao beans are infused with cannabis and mushroom compounds follows the processes described above and outlined in FIGS. 1-7. Once the cannabis and mushroom compounds infused cacao beans are obtained, they can be stored frozen to help preserve freshness and slow/prevent deterioration of the cannabis and mushroom compounds that reduces the efficacy of the cannabis and mushroom compounds.

The cannabis and mushroom compounds infused cacao beans can be processed in any of a variety of methods similar to standard cacao beans (which are not infused) to produce cannabis and mushroom compounds infused cacao and chocolate products, such as but not limited to, hot chocolate beverage, chocolate chips, chocolate bars, chocolate bars with nuts and fruit, chocolate bars with nougat, and so on. In a preferred embodiment, cannabis and mushroom compounds infused chocolate bars with nuts and/or dehydrated fruits are made with chocolate from cannabis and mushroom compounds infused cacao beans along with cannabis and mushroom compounds infused nuts and/or dehydrated fruits that are produced by the methods disclosed herein. Such chocolate bars made with cannabis and mushroom compounds infused chocolate and nuts and/or dehydrated fruits have a greater level of cannabis and mushroom compounds and therefore potency than chocolate bars made solely with cannabis and mushroom compounds infused chocolate with nuts and/or fruits that are not infused.

### Example III: Popcorn

Cannabis and mushroom compounds infused popcorn kernels may be made by infusing popcorn kernels with a cannabis and mushroom compounds infused food grade oil obtained as described above and outlined in FIGS. 1-7.

To infuse popcorn kernels with cannabis and mushroom compounds from a cannabis and mushroom compounds infused food grade oil, raw kernels are added to infused food grade oil at a temperature between 150° F. and 200° F. (preferably 170° F.) in the process described above and in FIG. 7. The infused kernels can then be used directly for popcorn or can be treated using methods that impart various flavors and/or seasoning to the popcorn. Infused kernels that are to be used directly for popcorn are typically stored in a cooking oil, typically at about a 1:1 kernels to oil ratio. The infused kernels can then be popped directly from the stored mixture.

### Example IV: Infused Dehydrated Fruit

The process by which dehydrated fruit is infused with cannabis and mushroom compounds follows the processes described above and outlined in FIGS. 1-7.

Virtually any dehydrated fruit can be used in the cannabis and mushroom compounds infusion process, such as, but not limited to, dehydrated strawberries, other berries, cherries, apricots, mangos, grapes (raisins), cranberries, prunes, and so forth. Once the cannabis and mushroom compounds infused dehydrated fruit is obtained, the fruit can be stored frozen to help preserve freshness and slow/prevent deterioration of the cannabis and mushroom compounds that reduces the efficacy of the cannabis and mushroom compounds. The cannabis and mushroom compounds infused dehydrated fruit can be consumed directly or used in the preparation of various foods that contain dehydrated fruit, such as granola bars, chocolate bars, baked goods, trail mix, and the like. When used in the preparation of other foods, the cannabis and mushroom compounds infused dehydrated fruit will provide a source of whole spectrum cannabis and mushroom compounds to such foods.

### Example V: Infused Raw Fruit

The process by which raw fruit is infused with cannabis and mushroom compounds generally follows the processes described above and outlined in FIGS. 1-7, with some modifications. In a preferred embodiment, MCT oil is used as the infused oil is used, given its relatively low melting/liquifying temperature.

To infuse raw fruit, the raw fruit is mixed with the cannabis and mushroom compounds infused oil at a temperature between about 100° F. and 150° F. for 15 minutes to one hour. The mixture of infused oil and fruit is then frozen solid. Following the freezing, the mixture is liquified at a temperature between about 100° F. and 150° F. and then the fruit is separated from the oil using an acceptable straining method, such as straining through a nylon mesh filter. Preferably, the type of raw fruit used in this process has a relatively high density that helps keep the fruit intact throughout the process. For example, pineapple and mango are fruits that have been used in this process to produce mushroom compounds infused pineapple and mango. The infused fruits can be stored frozen to help preserve the integrity of the cannabis and mushroom compounds. The infused fruits can be consumed directly or used in other foods, such as the aforementioned chocolate and granola bars.

### Example VI: Raw and Roasted Nuts and Seeds

Cannabis and mushroom compounds infused raw or roasted nuts and seeds may be made by infusing raw or roasted nuts and seeds with a cannabis and mushroom compounds infused food grade oil obtained as described above and outlined in FIGS. 1-7.

To infuse raw nuts and seeds or roasted nuts and seeds with cannabis and mushroom compounds from a cannabis and mushroom compounds infused oil, the consumables are added to infused oil at a temperature between 150° F. and 200° F. (preferably 170° F.) in the process described above and in FIG. 4. The infused nuts and seeds can then be consumed directly or used in other foods, such chocolate and granola bars, trail mix, and so forth.

### Example VII: Granola Bars

Cannabis and mushroom compounds infused granola bars can be made using suitable cannabis and mushroom compounds infused food grade oil as infused using the infusion processes described above and outlined in FIGS. 1-7.

Once a suitable cannabis and mushroom compounds infused oil is produced, the infused oil can be used as a base for granola bars. The bars can be made with a variety of cannabis and mushroom compounds infused nuts and/or seeds that are formed into granola bars with oatmeal (that likewise can be infused with cannabis and mushroom compounds) or a similar foodstuff that is commonly used in granola bars. Such granola bars formed in this manner can contain a significant number of cannabis and mushroom compounds.

### Example VIII: Infused Garlic and Beans

Cannabis and mushroom compounds infused garlic and beans (such as pinto beans, navy beans, and other similar beans) can be made using a suitable cannabis and mushroom compounds infused food grade oil infused with cannabis and mushroom compounds using the infusion processes described above and outlined in FIGS. 1-7.

The process by which garlic or beans are infused with cannabis and mushroom compounds closely follows the general process described above and outlined in FIG. 7. The heating steps described in the process 302-312 (FIG. 7) for infusing garlic and beans is typically carried out at a temperature between 150° F. and 170° F. for 3-4 hours. Once the cannabis and mushroom compounds infused garlic or beans are obtained, the garlic or beans can be stored frozen to help preserve freshness and slow/prevent deterioration of the cannabis and mushroom compounds that reduces the efficacy of the mushroom compounds.

### Example IX: Infused Bacon

Cannabis and mushroom compounds infused bacon can be made using a suitable cannabis and mushroom compounds infused good grade oil made with the oil infusion processes described above and outlined in FIGS. 1-7.

Once a suitable cannabis and mushroom compounds infused oil is produced, the infused oil can be used to infuse bacon with cannabis and mushroom compounds. To infuse bacon, raw bacon is initially laid out on a baking pan or similar baking sheet that preferably has raised edges. The bacon is then coated with the cannabis and mushroom compounds infused oil, along with a sufficient amount of infused oil to keep a layer of infused oil in the bottom of the baking pan to ensure that the bacon is exposed to the infused oil. The bacon with infused oil is then frozen solid. After freezing, the bacon with infused oil is heated to a temperature between 170° F. and 250° F. for 1-4 hours or until the bacon is crisp. The resulting cannabis and mushroom compounds infused bacon is then strained from the cannabis and mushroom compounds infused oil and resulting cannabis and mushroom compounds infused bacon grease (such that there is a cannabis and mushroom compounds infused oil and bacon grease mixture). The bacon is then stored for subsequent use, preferably either frozen or at a refrigerated/cool temperature.

The resulting cannabis and mushroom compounds infused oil and bacon grease mixture is a bacon flavored mixture that can be used in the methods disclosed herein to infuse consumables with cannabis and mushroom compounds and impart/add a bacon flavor to the consumable. For example, the mushroom compounds infused oil and bacon grease mixture can used to prepare popcorn as detailed above, resulting in a cannabis and mushroom compounds infused, bacon flavored popcorn.

### Example X: Infused, Flavored Oil

As detailed above, cannabis and mushroom compounds infused food grade oils can be produced using essentially all food grade oils and butter, including but not limited to, coconut oil, butter, MCT oil, shortening, lard, vegetable oil, sunflower oil, olive oil, grape seed oil, avocado oil, and combinations thereof. Any of these oils and butter can be infused with cannabis and mushroom compounds using the oil infusion processes described above and outlined in FIGS. 1-6. Cannabis and mushroom compounds infused oils and butter can be used to infuse various consumables by the methods described above and outlined in FIG. 7 as well as the various specific examples described herein, such as those for infused raw fruit, bacon, garlic, and so on. The methods described herein may also be used to produce infused cooking flavoring agents, such as herbs and spices. Such cannabis and mushroom compounds infused, flavoring agents (herbs and spices) can be used on food to provide cannabis and mushroom compounds and flavor.

Once a cannabis and mushroom compounds consumable is produced by the methods described herein, the residual cannabis and mushroom compounds infused food grade oil can typically be used in the process again since the residual infused oil still typically contains a significant amount of cannabis and mushroom compounds. In this manner, residual infused oil typically can be used in the process once or twice, such that infused oil can overall be used 2-3/several times in the process.

Another aspect of the residual cannabis and mushroom compounds infused oil is that the oil typically acquires/takes on flavor from the infused consumable. As indicated above, the process used to infuse bacon with cannabis and mushroom compounds produces a residual cannabis and mushroom compounds infused, bacon flavored oil and bacon grease mixture. The processes used to infuse other consumables likewise results in a flavored, cannabis and mushroom compounds infused oil. For example, the residual oil from the process used to produce cannabis and mushroom compounds infused garlic will have a garlic flavor; residual oil from cannabis and mushroom compounds infused fruit will have a fruit flavor; residual oil from cannabis and mushroom compounds infused with coffee will have a coffee flavor; and so on. The flavored, cannabis and mushroom compounds infused oils can be used to impart both flavors along with cannabis and mushroom compounds to other foods in several ways, such as, but not limited to, by directly using the infused oil on food, as a cooking oil, in the infusion methods disclosed herein, and so forth.

### Example XI: Ice Cream, Jams, and Jellies with Infused Fruit

As detailed above, cannabis and mushroom compounds infused fresh and dehydrated fruits can be produced by methods disclosed herein. Such cannabis and mushroom compounds infused fruits can in turn be used in ice cream, jams, and jellies to produce cannabis and mushroom compounds infused, flavored ice cream, jams, and jellies. For example, cannabis and mushroom compounds infused fresh fruit can be used to make a slurry that in turn can be used in making ice cream, jams, or jellies, resulting in cannabis and mushroom compounds infused and flavored ice cream, jams, or jellies. The cannabis and mushroom compounds content of such products can be increased by adding other cannabis and mushroom compounds infused consumables, such as cannabis and mushroom compounds infused nuts and/or chocolate.

### Example XII: Caramel Sauce Containing Infused Cannabis and Mushroom Compounds

As detailed above, cannabis and mushroom compounds infused food grade oil can be produced by the methods disclosed herein. Such oil can in turn be added to caramel sauce to produce a cannabis and mushroom compounds containing caramel sauce. A cannabis and mushroom compounds containing caramel sauce can be used in the production of ice cream to add cannabis and mushroom compounds into the ice cream. Alternatively, a cannabis and mushroom compounds containing caramel sauce can be used as a topping, a flavoring agent/ingredient for other foods, and so forth. In this regard, cannabis and mushroom compounds containing caramel sauce can be used to add both flavor along with cannabis and mushroom compounds to a variety of consumables.

### Example XIII: Cannabis and Mushroom Compounds Infused Baked Goods

As detailed above, cannabis and mushroom compounds infused food grade oil can be produced by the methods disclosed herein. Such infused oil can in turn be used to make various baked goods, such as cookies, brownies, quick breads, and so forth. In this regard, cannabis and mushroom compounds infused oil can be added as a base ingredient for baked goods, such as the food grade oil in baked goods or in combination with other base ingredients, such as butter. In one exemplary embodiment, a ratio of 75% cannabis and mushroom compounds infused oil to 25% cannabis and mushroom compounds infused butter is used as a base ingredient in making cookies, brownies, and other baked goods.

### Example XIV: Cannabis and Mushroom Compounds Infused Freeze Dried Consumables

As detailed above, cannabis and mushroom compounds infused food grade oil can be produced by the methods disclosed herein. Such infused oil can in turn be used to make a wide variety of various cannabis and mushroom compounds infused consumables, such as infused raw fruit, ice cream, and so forth. A wide variety of cannabis and mushroom compounds infused consumables can in turn be treated by the process of lyophilization, commonly known as freeze-drying, to turn the cannabis and mushroom compounds infused consumables into another form of consumable. Lyophilization typically removes 98% of the original water content, typically resulting in a crunchy and/or creamy consumable. Accordingly, the methods described herein can be applied to produce cannabis and mushroom compounds infused consumables that in turn are freeze-dried to produce freeze-dried cannabis and mushroom compounds infused consumables. The freeze-drying process allows foods such as infused fruit to retain both its cannabis and mushroom compounds and its nutrition.

### Example XV: Pressure Cooked Cannabis and Mushroom Compounds Infused Consumables

As detailed above, cannabis and mushroom compounds infused food grade oil can be produced by the methods disclosed herein. Such infused oil can in turn be used to make a wide variety of various cannabis and mushroom compounds infused consumables by methods that include heating and freezing steps to infuse the consumables with cannabis and mushroom compounds, as described above. In an exemplary embodiment, the heating steps may be carried out under pressure, such as in a pressure cooker. In this regard, the levels of infused cannabis and mushroom compounds in an infused consumable produced with pressure cooking can be greater than the relative levels obtained without pressure cooking. Accordingly, pressure cooking, or similar pressurized heating can be used to produce consumables that potentially have even higher levels of infused cannabis and mushroom compounds when compared to the infused consumables produced by the methods disclosed herein.

1. A method of producing a food grade oil infused with whole spectrum cannabis compounds and whole spectrum mushroom compounds, the method comprising:
infusing a food grade oil with whole spectrum cannabis compounds by:
(A) providing cannabis;
(B) freezing the cannabis to yield frozen cannabis;
(C) providing a food grade oil at a temperature of 170° F.-240° F.;
(D) adding at least a portion of the frozen cannabis of (B) to the food grade oil of (C) to form a cannabis and food grade oil mixture;
(E) heating the cannabis and food grade oil mixture of (D) to a temperature of 170° F.-240° F. for 4-12 hours;
(F) freezing the cannabis and food grade oil mixture of (E) into a solid, frozen biomass of cannabis and food grade oil mixture;
(G) heating the solid, frozen biomass of cannabis and food grade oil mixture of (F) at a temperature of 150° F.-200° F. for 2-4 hours;
(H) straining the cannabis and food grade oil mixture of (G) to separate the food grade oil from the cannabis to yield a whole spectrum cannabis compounds infused food grade oil;
(I) heating the cannabis infused food grade oil of (H) to a temperature of 250° F.-300° F. for 1-6 hours;
(J) cooling the cannabis infused food grade oil of (I) to a temperature of 150° F.-200° F. or storing the cannabis infused food grade oil for subsequent use;
infusing a food grade oil with whole spectrum mushroom compounds by:
(K) providing mushroom material;
(L) providing a food grade oil at a temperature of 170° F.-240° F.;
(M) adding at least a portion of the mushroom material of (K) to the food grade oil of (L) to form a mushroom material and food grade oil mixture;
(N) heating the mushroom material and food grade oil mixture of (M) to a temperature of 170° F.-240° F. for 4-12 hours;
(O) freezing the mushroom material and food grade oil mixture of (N) into a solid, frozen biomass;
(P) heating the solid, frozen biomass of mushroom material and food grade oil mixture of (O) at a temperature of 150° F.-200° F. for 2-4 hours;
(Q) straining the mushroom material and food grade oil mixture of (P) to separate the food grade oil from the mushroom material to yield a whole spectrum mushroom compounds infused food grade oil; and
combining the whole spectrum cannabis compounds infused food grade oil of (J) with the whole spectrum mushroom compounds infused food grade oil of (Q) to yield a food grade oil infused with whole spectrum cannabis compounds and whole spectrum mushroom compounds.

2. The method of claim 1 further comprising: repeating steps (E) and (F) one or more times before step (G).

3. The method of claim 1 further comprising: repeating steps (N) and (O) one or more times before step (P).

4. The method of claim 1 further comprising: repeating steps (E) and (F) one or more times before step (G); and
repeating steps (N) and (O) one or more times before step (P).

5. The method of claim 1, wherein the food grade oil provided in (C) and (L) is selected from the group consisting of coconut oil, medium chain triglycerides oil, vegetable oil, sunflower oil, olive oil, grape seed oil, avocado oil, butter, shortening, margarine, lard, and a combination thereof.

6. The method of claim 1, wherein the food grade oil provided in (C) and (L) is coconut oil.

7. The method of claim 1, wherein the cannabis provided in (A) is selected from the group consisting of Cannabis Sativa, Cannabis indica, and Cannabis Ruderalis.

8. The method of claim 1, wherein the mushroom material in (K) is obtained from mushrooms selected from the group consisting of chanterelle, truffle, morel, trumpet, oyster, shiitake, maitake, cremini, lobster, porcini, hedgehog, portobello, white, button, eenoki, cordyceps, lion's mane, reishi, argarikon, cordyceps, chaga, maitake, turkey tail, shiitake, psilocybin, and combinations thereof.

9. A method of infusing an orally ingestible consumable with whole spectrum cannabis compounds and whole spectrum mushroom compounds, the method comprising:
infusing a food grade oil with whole spectrum cannabis compounds by:
(A) providing cannabis;
(B) freezing the cannabis to yield frozen cannabis;
(C) providing a food grade oil at a temperature of 170° F.-240° F.;
(D) adding at least a portion of the frozen cannabis of (B) to the food grade oil of (C) to form a cannabis and food grade oil mixture;
(E) heating the cannabis and food grade oil mixture of (D) to a temperature of 170° F.-240° F. for 4-12 hours;
(F) freezing the cannabis and food grade oil mixture of (E) into a solid, frozen biomass of cannabis and food grade oil mixture;
(G) heating the solid, frozen biomass of cannabis and food grade oil mixture of (F) at a temperature of 150° F.-200° F. for 2-4 hours;
(H) straining the cannabis and food grade oil mixture of (G) to separate the food grade oil from the cannabis to yield a whole spectrum cannabis compounds infused food grade oil;
(I) heating the cannabis infused food grade oil of (H) to a temperature of 250° F.-300° F. for 1-6 hours;
(J) cooling the cannabis infused food grade oil of (I) to a temperature of 150° F.-200° F. or storing the cannabis infused food grade oil for subsequent use;
infusing a food grade oil with whole spectrum mushroom compounds by:
(K) providing mushroom material;
(L) providing a food grade oil at a temperature of 170° F.-240° F.;
(M) adding at least a portion of the mushroom material of (K) to the food grade oil of (L) to form a mushroom material and food grade oil mixture;
(N) heating the mushroom material and food grade oil mixture of (M) to a temperature of 170° F.-240° F. for 4-12 hours;
(O) freezing the mushroom material and food grade oil mixture of (N) into a solid, frozen biomass;
(P) heating the solid, frozen biomass of mushroom material and food grade oil mixture of (O) at a temperature of 150° F.-200° F. for 2-4 hours;
(Q) and straining the mushroom material and food grade oil mixture of (P) to separate the food grade oil from the mushroom material to yield a whole spectrum mushroom compounds infused food grade oil;
(R) combining the whole spectrum cannabis compounds infused food grade oil of (J) with the whole spectrum mushroom compounds infused food grade oil of (Q) to yield a food grade oil infused with whole spectrum cannabis compounds and whole spectrum mushroom compounds;
(S) adding at least one orally ingestible consumable to the whole spectrum cannabis compounds and whole spectrum mushroom compounds infused food grade oil of (R) to form a consumable and infused oil mixture;
(T) heating the consumable and infused oil mixture of (S) to a temperature of 170° F.-240° F. for 4-12 hours;
(U) freezing the consumable and infused oil mixture of (T) to form a solid, frozen mixture of consumable and infused oil;
(V) heating the solid, frozen mixture of consumable and infused oil of (U) to a temperature of 150° F.-200° F. for 2-4 hours to liquefy the infused oil; and
(W) straining the consumable from the infused oil of (V) to yield a consumable infused with whole spectrum cannabis compounds and whole spectrum mushroom compounds.

10. The method of claim 9, further comprising: (X) freezing the infused consumable of (W).

11. The method of claim 9 further comprising: repeating steps (E) and (F) one or more times before step (G).

12. The method of claim 9 further comprising: repeating steps (N) and (O) one or more times before step (P).

13. The method of claim 9 further comprising: repeating steps (T) and (U) one or more times before step (V).

14. The method of claim 9 further comprising: repeating steps (E) and (F) one or more times before step (G);
repeating steps (N) and (O) one or more times before step (P); and
repeating steps (T) and (U) one or more times before step (V).

15. The method of claim 9, wherein the food grade oil provided in (C) and (L) is selected from the group consisting of coconut oil, medium chain triglycerides oil, vegetable oil, sunflower oil, olive oil, grape seed oil, avocado oil, butter, shortening, margarine, lard, and a combination thereof.

16. The method of claim 9, wherein the food grade oil provided in (C) and (L) is coconut oil.

17. The method of claim 9, wherein the cannabis provided in (A) is selected from the group consisting of Cannabis Sativa, Cannabis Indica, and Cannabis Ruderalis.

18. The method of claim 9, wherein the mushroom material in (K) is obtained from mushrooms selected from the group consisting of chanterelle, truffle, morel, trumpet, oyster, shiitake, maitake, cremini, lobster, porcini, hedgehog, portobello, white, button, eenoki, cordyceps, lion's mane, reishi, argarikon, cordyceps, chaga, maitake, turkey tail, shiitake, psilocybin, and combinations thereof.

19. The method of claim 9, wherein the consumable is selected from the group consisting of coffee beans, cacao beans, popcorn kernels, raw nuts, raw seeds, roasted nuts, roasted seeds, and dehydrated fruit.

20. The method of claim 9, wherein the consumable is coffee beans.
